(19) United States
(12) Patent Application Publication
(10) Pub. No.: US 2003/0130606 A1
(43) Pub. Date: Jul. 10, 2003

(54) Blood treatment replacement fluid using infusible fluids in combination

(76) Inventor: Joseph E. Tuck, North Andover, MA (US)

Correspondence Address: PROSKAUER ROSE LLP, PATENT DEPARTMENT, 1585 BROADWAY, NEW YORK, NY, 10036, US

(21) Appl. No.: 10/040,659

(22) Filed: Jan. 7, 2002

(51) Int. Cl. A61M 37/00; C02F 1/44

(52) U.S. Cl. 604/4.01; 210/645

(57) ABSTRACT

An infusible replacement fluid does not require separate regulatory clearance and does not need to be prepared by a pharmacist. The fluid is made from constituent fluids that are cleared for infusion. Further, the fluids are available in standard volumes of such size that that the proper proportions may be provided by a convenient number and size of packages. In a preferred embodiment, the replacement fluid is provided to the patient by “hanging” five bags of fluid, one of half-normal saline (e.g., 1 liter bag), three of ringers solution (e.g., 3 liter bags), and one sodium lactate for injection (e.g., 1 liter bag). The resulting concentrations of solutes closely approximates an ideal solution. The above constituents may be purchased and stored in a convenient location and used as required without the need for a pharmacist to prepare the fluids.

### FIELD OF THE INVENTION

The invention relates to injectable replacement fluid for use in blood treatments such as hemofiltration and hemodiafiltration, etc. and more particularly to such replacement fluids that consist of constituent components that are infusible independently. For example, the constituents may have independently qualified for governmental regulatory clearance or may be known to be independently medically-sound for infusion.

### BACKGROUND

There are many types of blood processing and fluid exchange procedures, each providing different therapeutic effects and demanding different processing criteria. Typically, such procedures entail the removal of blood or another fluid from an individual and the return of blood or another fluid to the individual in a controlled fashion. Examples of such procedures include hemofiltration (HF), hemodialysis (HD), hemodialysis with hemofiltration (HDF), and peritoneal dialysis (PD).

Typically, when performing the blood processing and fluid exchange procedures of the type just described, a replacement or make-up fluid is returned back to the patient in some proportion to the amount of fluid that is removed from the individual. The type and make-up of fluids that these procedures handle vary according to the particular treatment modality being performed, e.g., among waste fluid and replacement fluid in hemofiltration or hemodiafiltration; or replacement fluid and dialysis solution in hemodialysis or hemodiafiltration); or fresh peritoneal dialysis solution and spent peritoneal dialysis solution in peritoneal dialysis.

Frequently replacement fluid is prepared by a pharmacist according to a prescription. In fact, in certain circumstances, manufactured infusible fluids may not be available at all. This may be true, for example where no manufacturer has incurred the necessary expense and effort to obtain regulatory clearance. This problem of the economics of obtaining regulatory clearance is exacerbated by the fractionation of the markets for such fluids. That is, the correct balance of electrolytes and respective concentrations depend on the type of treatment. For hemofiltration and hemodiafiltration, acceptable electrolyte mixtures are well known. One example solution for use as a replacement fluid has the following constituents:

| Constituents | | Phosphorous-based | Calcium-based |
|---|---|---|---|
| NaCl | (mEq/l)* | 60-100* | 60-100* |
| NaHCO3 | (mEq/l) | 80-40* | 80-40* |
| KCl | (mEq/l) | 2 | 2-4 |
| K3PO4 | (mEq/l) | 2 | 0 |
| MgSO4 | (Meq/L) | 0.5-1.5 | 0.5-1.5 |
| Dextrose | (g/l) | 0-2.0 (0-0.2%) | 0-0.2 (0-0.2) |
| CaCl2 | (mEq/l) | 0 | 3-4** |

Pharmacy-prepared replacement fluid solutions are expensive. Premanufactured solutions would be much more desirable, however, the regulatory clearance process is long and expensive. At present, the cost of providing replacement fluid is driven by the cost of preparation by a pharmacist. In large treatment facilities, economies of scale may keep such costs down. In smaller settings that require only relatively small stocks, the cost of preparation of replacement fluid by a pharmacist multiples the cost of the fluids many fold.

### SUMMARY OF THE INVENTION

According to the present invention, an infusible replacement fluid does not require separate regulatory clearance and that does not need to be prepared by a pharmacist. The fluid is made from constituent fluids that are appropriate for infusion. The fluids may be available in standard volumes of such size that that the proper proportions may be provided by a convenient number and size of packages. In a preferred embodiment, the replacement fluid is provided to the patient by “hanging” five bags of fluid, one of half-normal saline (e.g., 1 liter bag), three of ringers solution (e.g., 3 liter bags), and one sodium lactate for injection (e.g., 1 liter bag). The resulting concentrations of solutes closely approximates an ideal solution. The above constituents may be purchased and stored in a convenient location and used as required without the −15 need for a pharmacist to prepare the fluids.

The invention will be described in connection with certain preferred embodiments, with reference to the following illustrative figures so that it may be more fully understood. With reference to the figures, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

FIG. 1 is a figurative illustration of a blood treatment system with a supply of replacement fluid provided by five standard containers of infusible fluids in such combination as to provide replacement fluid for a hemofiltration or other extracorporeal blood treatment requiring replacement of fluid.

FIG. 2 is an illustration for purposes of describing a method for making a container of replacement fluid from stock fluids.

FIG. 3 is a figurative illustration of a blood treatment system with a supply of replacement fluid provided by a container filled with a fluid composed of infusible fluids in such combination as to provide replacement fluid for a hemofiltration or other extracoproreal blood treatment requiring replacement of fluid.

FIG. 4 is an exploded illustration of a blood treatment machine packaged with instructions for use including instructions for providing a replacement fluid using fluids for injection in combination.

FIG. 5 is an exploded illustration of a tubing set for blood treatment packaged with instructions for use including instructions for providing a replacement fluid using fluids for injection in combination.

FIG. 6 is an exploded illustration of a manifold packaged with instructions for use including instructions for providing a replacement fluid using fluids for injection in combination.

FIG. 7 is an exploded illustration of large fluid containers for mixing constituents that are packaged with instructions for use including instructions for providing a replacement fluid using fluids for injection in combination.

FIG. 8 is an illustration of a case with instructions for preparing replacement fluid packaged with constituent components.

FIG. 9 shows a manifold with a figurative illustration of a fluid property sensor.

FIG. 10 illustrates a flow control system that mixes defined proportions of fluid to generate a replacement fluid.

### DETAILED DESCRIPTION OF THE PREFERRED EMBODIMENTS

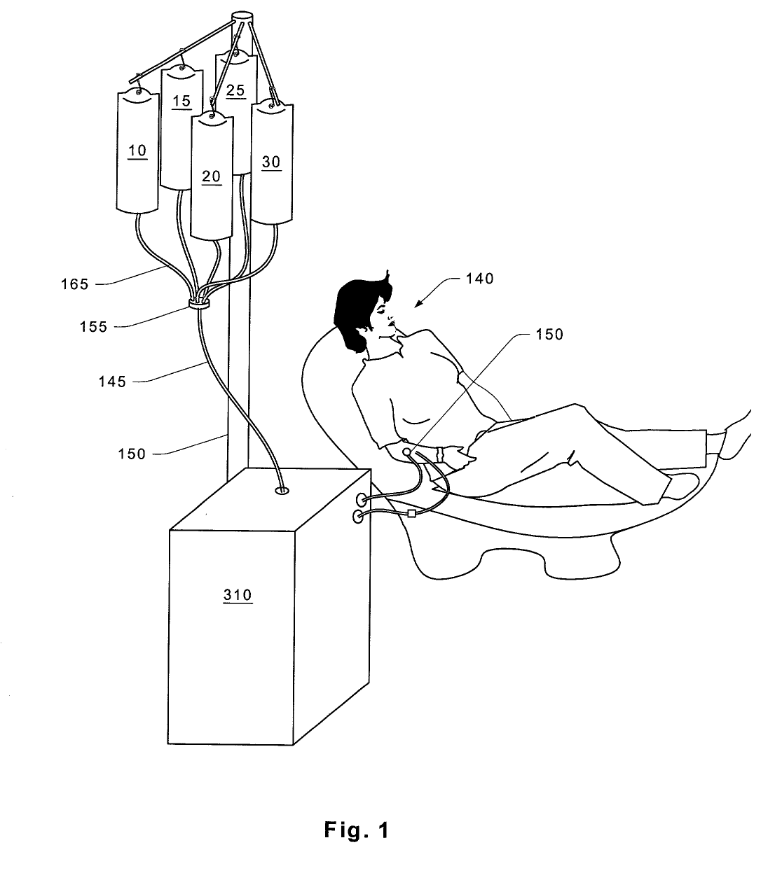

Referring to FIG. 1, a patient 140 is receives a blood treatment by a continuous process performed by a blood treatment machine 310. The process extracts fluid from the blood of the patient 140 which must be replaced to prevent the patient 140 from dehydrating. For example, the treatment process may be hemofiltration or hemodiafiltration. In such processes, blood is drawn from the patient 140 through an access 150 and returned to the patient 140 through the same access 150.

As is known in the art, the treatment process provided by the blood treatment machine may remove substantial quantities of fluids including electrolytes from the patent's 140 blood. As part of the process, as is also known, fluid may be provided to the patient 140 during treatment. To prevent the patient being poisoned by the replacement fluid, however, it must be an admixture that includes certain electrolytes in the correct balance to insure that what is lost is replaced without depleting the patient of essential nutrients. During hemofiltration, for example, multiple liters of fluid may be required to replace what is withdrawn from the patient during treatment. In the prior art, such replacement fluid is prescribed by a physician and prepared by a pharmacist.

The blood treatment machine 310 may be an automated device designed for use without continuous monitoring by skilled personnel in a setting such as a home or hospice. In such a setting, a user may require a small stock of replacement fluid for multiple treatments, which would not be nearly as great as would be required by a professional treatment facility having a capacity to treat many patients simultaneously.

As a result, obtaining a stock of prescription replacement fluid may be very expensive because of the professional labor involved in its preparation. To avoid the expense of providing replacement fluid from a pharmacist, a set of off-the-shelf replacement fluids may be combined. In FIG. 1, standard containers each containing one of three types of fluid, feed into a header 155. In a preferred embodiment, each fluid is regulatory-cleared for injection and the desired admixture suitable as a replacement fluid consists of:

1. One part half-normal saline (0.45% NaCL)

2. Three parts Ringers for injection (Na 147.5 mEq/L; Ca 4.5 mEq/L; K 4 mEq/L; and Cl 156 mEq/L) and

3. One part sodium lactate for injection (Na 167 mEq/L and Lactate 167 mEq/L).

This admixture can be generated on the fly by hanging standard containers (e.g. 1L each), from a standard IV support 160 for example, and supplying them directly to the blood treatment machine 310 through respective lead lines 165 that merge in the header 155.

The combined fluid is supplied through a single line 145 to the treatment machine 310. Since each fluid is appropriate for injection and available in standard quantities, they may be provided according to prescription without the need for a pharmacist to prepare a complete replacement fluid. Although each is incomplete on its own to provide a proper replacement fluid, they may be combined conveniently as shown to provide the correct admixture during treatment.

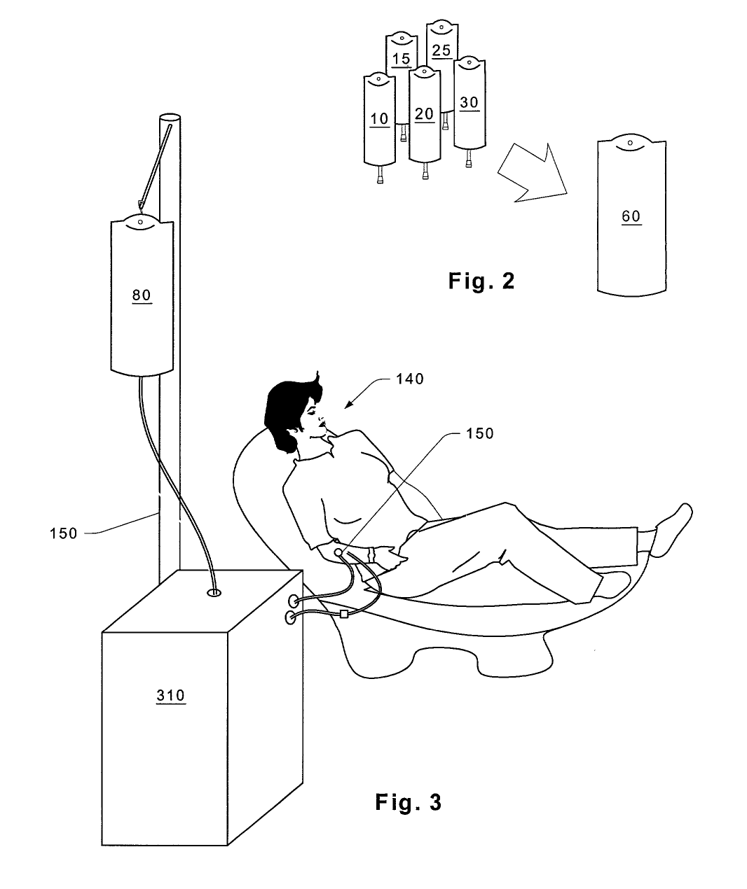

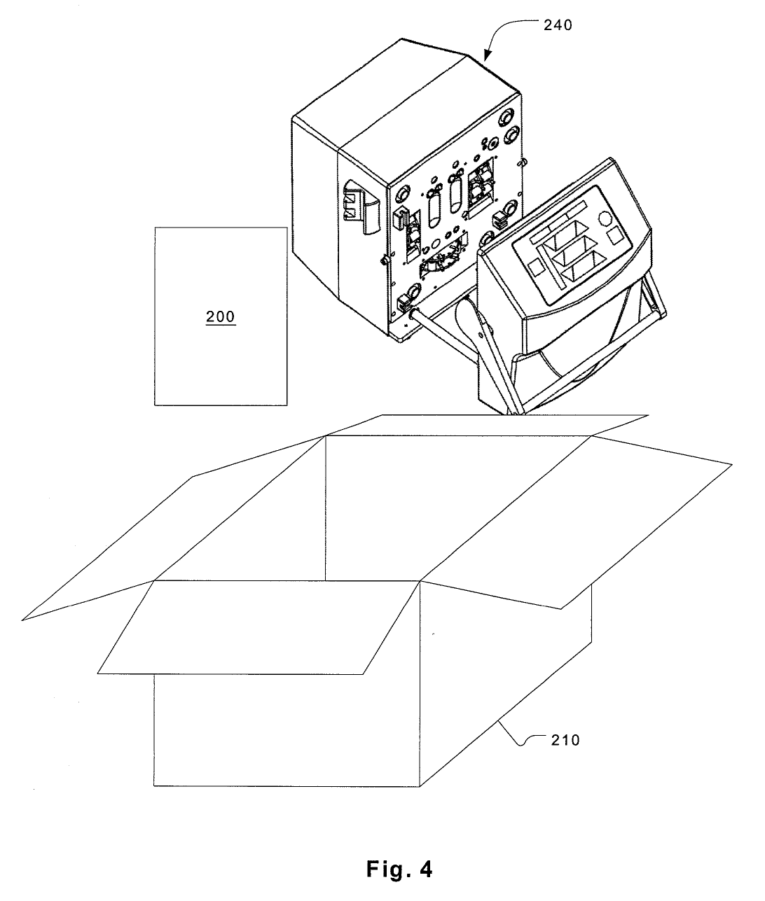

Referring to FIGS. 2 and 3, the proper admixture for replacement fluid may be provided, according to an alternative embodiment, by combining the above-mentioned constituents prior to administration through a blood processing machine 310. Here, a large container 60 is filled with the contents of all the above-mentioned constituents 10-30 in the noted proportions to obtain a single container of complete replacement fluid 80 which is then connected as shown to supply replacement fluid during treatment.

Referring to FIG. 4, instructions for providing replacement fluid according to either of the embodiments represented in FIGS. 1-3 may be incorporated in a printed enclosure 200 that may be packaged with a blood treatment machine 240. The machine 240 and printed enclosure 200 are placed in the same shipping container 210. As a result, a customer (e.g., patient, physician, nurse, etc.) receiving the blood treatment machine may learn to use the low-cost convenient method of the invention for providing replacement fluid. The printed enclosure 200 may be a user manual for the blood processing machine 240 that contains a portion providing the instructions for use of the inventive method. In an alternative embodiment, the printed enclosure 200 may contain a pointer to a network address or Internet site from which the instructions for the inventive method may be learned by a customer.

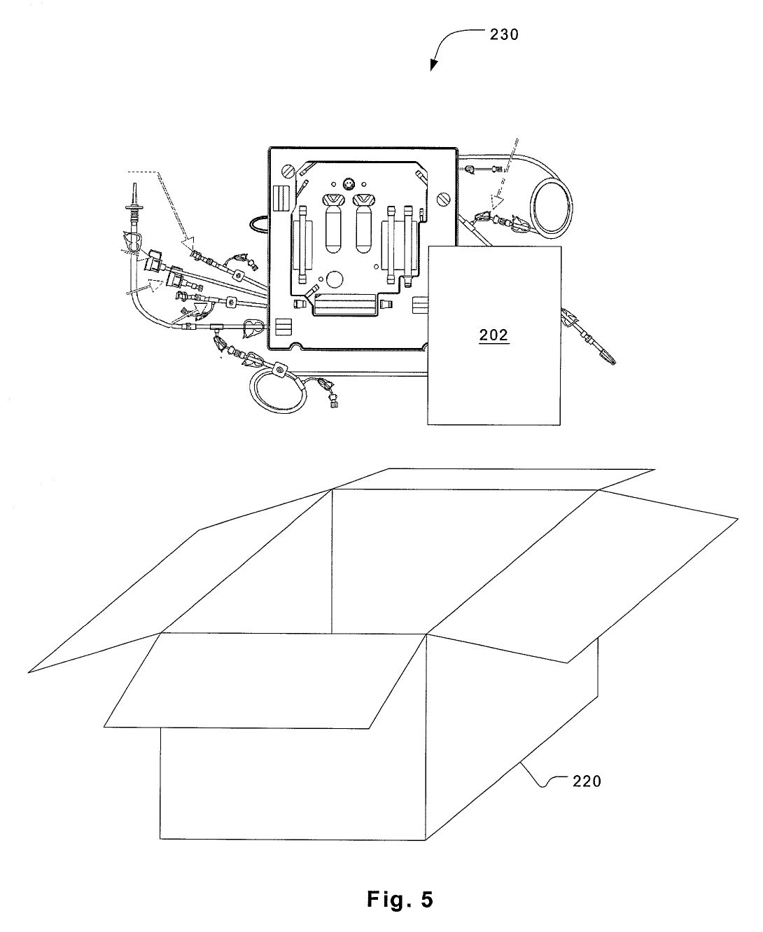

Referring to FIG. 5, instructions for providing replacement fluid according to either of the embodiments of FIGS. 1-3 may be incorporated in a printed enclosure 200 that may be packaged with a tubing set 230 that is ordinarily used as a replaceable part of blood processing machine (for example, 310 of FIG. 1). The tubing set (or multiple tubing sets) which may be disposable is placed together with the printed enclosure 200 into the same shipping container 220. As a result, a customer receiving the tubing set may learn to use the low-cost convenient method of the invention for providing replacement fluid. Again, the printed enclosure 210 may be a user manual for the blood processing machine (e.g., 240 or 310) and/or the tubing set 230, with a portion providing the instructions for use of the inventive method or a pointer to a network address or Internet site from which the instructions for the inventive method may be learned by a customer.

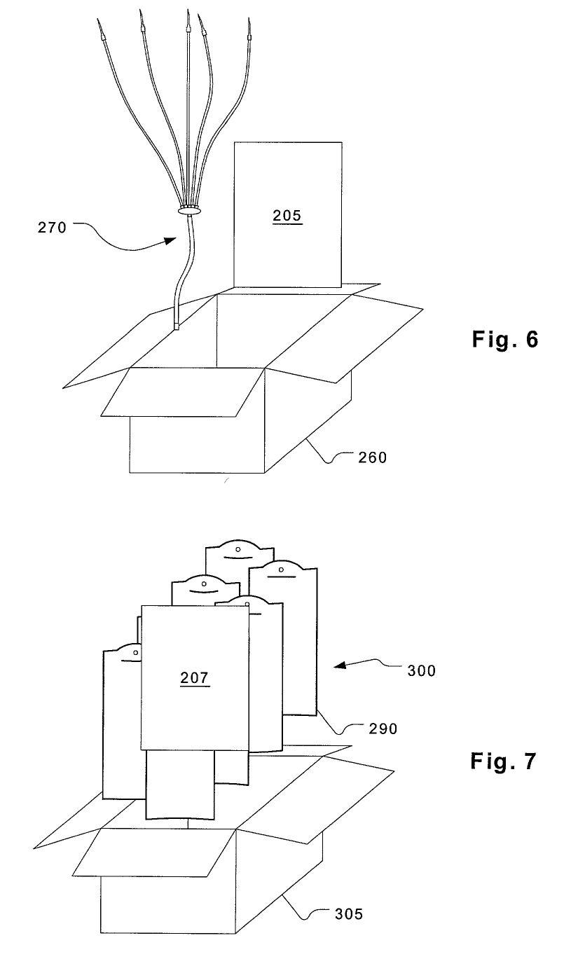

Referring now to FIG. 6, a header 270, such as may be used to practice the method illustrated in FIG. 1, may be packaged with a document 205 providing instructions for use of the method. Illustrated is an exploded view of a shipping container 260 which may contain one or more headers 270, which corresponds to header 155 in FIG. 1. The header 270 may be disposable. As a result of the combined packaging, a customer receiving the manifold may understand the low-cost convenient method of the invention for providing replacement fluid. Again, the printed enclosure 205 may be a user manual for the manifold 270 with a portion providing the instructions for use of the inventive method or a pointer to a network address or Internet site from which the instructions for the inventive method may be learned by a customer. Referring now to FIG. 7, a package may contain special or standard containers 300, each 290 adapted for use in creating and delivering the admixture as illustrated in the embodiment of FIG. 3. That is, each container 290 corresponds to an empty one of container 60 in FIGS. 2 and 80 in FIG. 3. Instructions 207 for preparing a replacement fluid in accord with the method of the invention may be provided in the same shipping container 305 used to package the containers 300. As discussed, the instructions 207 may be printed, pointed-to, embedded in another document, or multimedia instructions.

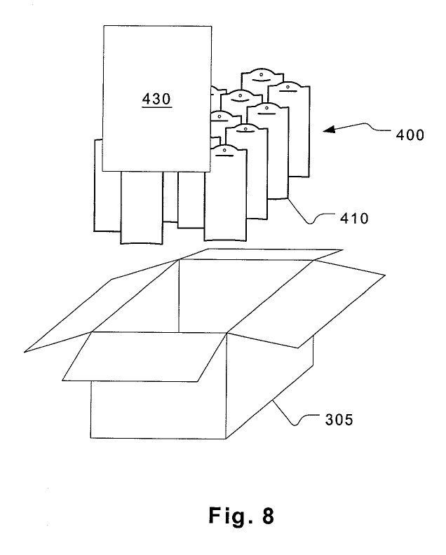

Referring now to FIG. 8, a package may contain standard containers 400, each 410 containing one of the constituents needed to form a complete replacement fluid for use in creating and delivering the admixture as illustrated in the embodiments of FIGS. 1-3 or others consistent with the invention. That is, each container 290 corresponds to one of the containers 10, 15, 20, 25, and 30 in FIGS. 1 and 2. Instructions 430 for utilizing the containers 400 to supply replacement fluid in accord with the method of the invention may be provided in the same shipping container 305 used to package the containers 400. As discussed above, the instructions 430 may be printed, pointed-to, embedded in another document, or multimedia instructions.

Note that although the invention has been described as being particularly suited for use in small-scale treatment facilities such as individual homes or hospices, it should be clear that the same benefits may accrue in larger treatment facilities despite the economies of scale.

Also, although the above embodiments discuss providing user instructions for a method of providing a replacement fluid in the form of a printed manuscript or point to an online resource, it is also possible to provide the instructions in alternative media. These include broadcast or recorded video, software training materials, interactive multimedia publications etc. All are regarded as being in scope of the invention.

Note that although in the above embodiments, the replacement fluid is formed without providing any further materials than the basic components, it is possible to modify the procedure while retaining the benefits of the invention. For example, small quantities of other fluids may be added without substantially modifying the appropriate proportions provided by the combination of constituent fluids. Thus, a container of half-normal saline solution, 3 containers of ringers solution, and a container of sodium lactate for injection could be combined through a manifold with some small amount of dextrose and administered as a replacement fluid. The proportions in this case would not be very upset by the addition of the dextrose solution and the resulting fluid would still qualify as an adequate replacement fluid. Thus, the combination of fluids according to the invention is not a rigid one and variations in consonance with the claims are possible.

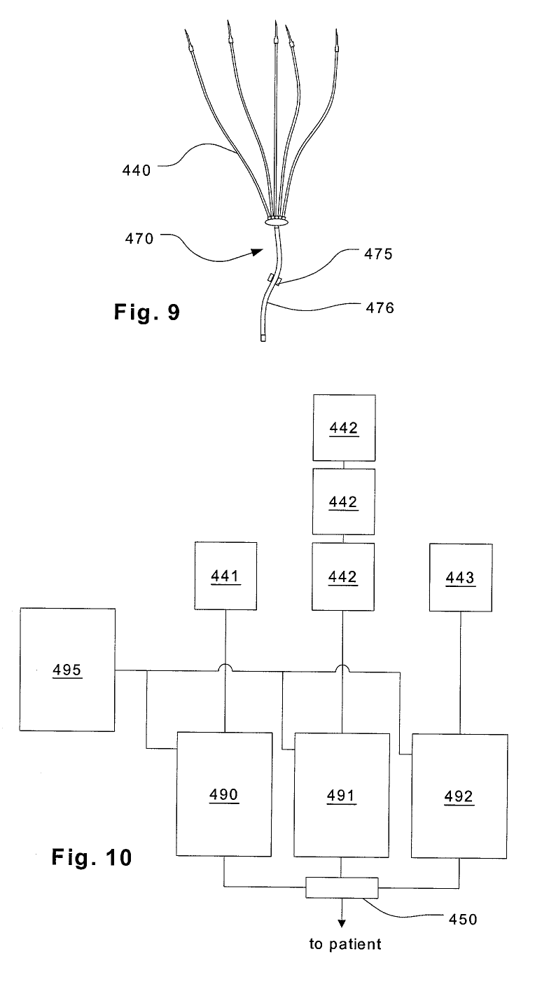

Referring now to FIG. 9, one way to ensure that the mixture obtained via a manifold 470 has the correct balance of components, is to employ a sensor 475 such as a conductivity cell to monitor the contents of the combined replacement fluid line 476. When the solutions are mixed in unbalanced proportions, such as due to a kink in one of the lines 440, the sensor may indicate this and trigger a shutdown of the blood treatment machine 310 and an alarm. The appropriate controls may be incorporated in the blood processing machine 310 and are not shown in detail, but may be implemented using known techniques. The sensor 475 may be a contact or non-contact device and may measure any appropriate property that would indicate whether the constituents are in balanced proportion or not. Alternative sensors include conductivity sensors, density sensors, radiation sensors (assuming components are radio-labeled), density sensors, etc.

Another mechanism for guaranteeing constituents are mixed in the proper proportion is to employ respective infusion pumps under common control. Referring now to FIG. 10, infusion pumps 490, 491, and 492 draw from respective containers 441, 442, and 443. Each pump is controlled by a common control 495 to pump at a rate that insures the correct admixture ratio is maintained. The pumping rates are preferably such consistent with the desired infusion rate demanded by the blood processing machine 310. The controller 495 may be part of the blood processing machine 310.

Note that by employing pumps controlled as indicated in FIG. 10, the constituents do not need to be in integral ratio containers. In fact, concentrated constituents can be diluted with half-normal saline or water. Note also that constituents can be measured and combined into a larger container as in the embodiment of FIGS. 2 and 3. In the latter case, the constituents may need to be measured to achieve the required proportions.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

We claim:

1. A method of delivering a replacement fluid to a patient undergoing a predefined blood extracorporeal blood treatment, comprising the steps of:
purchasing multiple infusible fluids each in a respective container and all being in such numbers and volumes as to form, when combined, an admixture defining an isotonic replacement fluid appropriate for said patient undergoing said predefined blood extracorporeal blood treatment;
said volumes being integral numbers of volumes of standard containers used for packaging said infusible fluids;
combining said multiple fluids and supplying it for infusion into said patient.

2. A method as in claim 1, wherein said step of purchasing includes purchasing at least one container of half-normal saline solution containing a total volume of 1 unit, purchasing at least one second volume container of ringers solution containing a total volume of 3 units, and purchasing at least one third volume container of sodium lactate for injection containing a total volume of 1 unit.

3. A method as in claim 1, wherein said half-normal saline is substantially 0.45% NaCL, said Ringers for injection is substantially Na 147.5 mEq/L; Ca 4.5 mEq/L; K 4 mEq/L; and Cl 156 mEq/L); and said sodium lactate for injection is substantially Na 167 mEq/L and Lactate 167 mEq/L.

4. A method as in claim 1, wherein said step of combining includes connecting said respective containers to a fluid line connected for infusion into said patient.

5. A method as in claim 4, wherein said step of connecting includes flowing fluids through a manifold and into said fluid line.

6. A method as in claim 1, wherein said step of combining includes flowing fluid from said respective containers into a common container and said step of supplying includes connecting said common container to a fluid line for infiltration into a patient.

7. A method of providing a replacement fluid for infusing a patient during an extracorporeal blood treatment that causes a loss of water and electrolytes from said patient, comprising the steps of:
obtaining respective containers of electrolyte/water mixtures that possess a property that makes them appropriate for injection;
combining contents of said containers in such proportion as to form a properly balanced replacement fluid;
supplying said replacement fluid to a patient during a treatment;
said step of combining taking place at a location substantially the same as said step of supplying.

8. A method as in claim 7, wherein said step of obtaining includes obtaining at least one container of half-normal saline solution containing a total volume of 1 unit, obtaining at least one second volume container of ringers solution containing a total volume of 3 units, and obtaining at least one third volume container of sodium lactate for injection containing a total volume of 1 unit.

9. A method as in claim 7, wherein said half-normal saline is substantially 0.45% NaCL, said Ringers for injection is substantially Na 147.5 mEq/L; Ca 4.5 mEq/L; K 4 mEq/L; and Cl 156 mEq/L); and said sodium lactate for injection is substantially Na 167 mEq/L and Lactate 167 mEq/L.

10. A method as in claim 7, wherein said step of combining includes connecting said respective containers to a fluid line connected for infusion into said patient.

11. A method as in claim 10, wherein said step of connecting includes flowing fluids through a manifold and into said fluid line.

12. A method as in claim 7, wherein said step of combining includes flowing fluid from said respective containers into a common container and said step of supplying includes connecting said common container to a fluid line for infiltration into a patient.

13. A method as in claim 7, wherein each of said electrolyte/water mixtures is individually regulatory-cleared for injection.

14. A package of materials for performing an extracorporeal blood treatment, comprising:
packaging materials enclosing:
at least one of a blood processing machine, a tubing set for use in a blood processing machine, a container for holding replacement fluid, a manifold for combining fluids from multiple containers, and a set of containers containing components that when combined define a replacement fluid;
at least one document describing or pointing to instructions for a method of providing a replacement fluid, the method including the steps of:
obtaining multiple infusible fluids each in a respective container and all being in such numbers and volumes as to form, when combined, an admixture defining an isotonic replacement fluid appropriate for said patient undergoing said predefined blood extracorporeal blood treatment;
said volumes being integral numbers of volumes of standard containers used for packaging said infusible fluids.

15. A package as in claim 14, wherein said method further includes the step of combining contents of said respective containers via a manifold with inlets for each of said respective containers and an outlet for said replacement fluid admixture.

16. A package as in claim 14, wherein said method further includes the step of combining contents of said respective containers in a single container connectable to supply replacement fluid to a patient.

17. A package as in claim 14, wherein instructions defining said method indicate manufacturer part numbers for said respective containers.

18. A package as in claim 14, wherein said step of obtaining includes obtaining containers of fluids that are regulatory-cleared for infusion.

19. A package as in claim 18, wherein instructions defining said method indicate manufacturer part numbers for said respective containers.

20. A package as in claim 14, wherein said step of obtaining includes obtaining containers of half-normal saline solution containing a total volume of 1 unit, obtaining containers of ringers solution containing a total volume of 3 units, and obtaining containers of sodium lactate for injection containing a total volume of 1 unit.

21. A package as in claim 20, wherein said half-normal saline is substantially 0.45% NaCL, said Ringers for injection is substantially Na 147.5 mEq/L; Ca 4.5 mEq/L; K 4 mEq/L; and Cl 156 mEq/L); and said sodium lactate for injection is substantially Na 167 mEq/L and Lactate 167 mEq/L.

22. A package as in claim 14, wherein said method wherein said method further includes the step of combining contents of said respective containers at a location that is the same as a location for treating said patient.

23. A method of infusing replacement fluid into a patient undergoing continuous extracorporeal blood treatment, comprising the steps of:
providing at least one standard volume container of half-normal saline solution containing a total volume of 1 unit;
providing at least one second standard volume container of ringers solution containing a total volume of 3 units;
providing at least one third standard volume container of sodium lactate for injection containing a total volume of 1 unit;
said half-normal saline, said ringers solution, and said sodium lactate for injection being regulatory cleared fluids for injection;
combining said at least one first, said at least one second, and said at least one third standard volume containers in situ for infusion into said patient during a blood treatment procedure requiring fluid replacement.

24. A method as in claim 23, wherein said step of combining includes flowing component fluids into a header from which a combined fluid is provided to a blood processing machine.

25. A method as in claim 23, wherein said step of combining includes transferring containers of different fluids into a single vessel and flowing fluid from said single vessel into a blood processing machine for infusion into a patient.

26. A method as in claim 23, wherein said containers contain, in combination the following proportionate quantities of solutions:
one part half-normal saline (0.45% NaCL);
three parts Ringers for injection (Na 147.5 mEq/L; Ca 4.5 mEq/L; K 4 mEq/L; and Cl 156 mEq/L); and
one part sodium lactate for injection (Na 167 mEq/L and Lactate 167 mEq/L).

27. A package of materials for performing an extracorporeal blood treatment, comprising:
at least one of a blood processing machine, a tubing set for use in a blood processing machine, a manifold for combining fluids from multiple containers, and a set of containers containing components that when combined define a replacement fluid;
at least one document describing or pointing to instructions for a method of providing a replacement fluid, the method including the steps of:
obtaining respective containers of electrolyte/water mixtures that possess a property that makes them appropriate for injection;
combining contents of said containers in such proportion as to form a properly balanced replacement fluid;
supplying said replacement fluid to a patient during a treatment;
said step of combining taking place at a location substantially the same as said step of supplying.

28. A method of delivering a replacement fluid, comprising the steps of:
providing an infusion controller configured to deliver fluids through multiple lines in a predetermined proportion;
connecting to said infusion controller lines from multiple containers each containing an infusate which when combined in accord with said predetermined proportion forms a physiologically-correct replacement fluid for replacing fluids lost during a predefined blood treatment;
combining the flows through said multiple lines into an infusion flow and delivering the same to a patient during a blood treatment process.

29. A method as in claim 28, wherein said step of connecting includes connecting said lines from multiple containers to at least two infusion pumps.

30. A method as in claim 28, wherein said step of connecting includes connecting said lines from multiple containers to at least two infusion pumps controlled by a common control signal.
